(19) United States
(12) Patent Application Publication
(10) Pub. No.: US 2008/0312661 A1
(43) Pub. Date: Dec. 18, 2008

(54) Lens Injector Lumen Tip for Wound Assisted Delivery

(76) Inventors: David A. Downer, Fort Worth, TX (US); Khiun F. Tjia, Epe (NL)

Correspondence Address: ALCON, IP LEGAL, TB4-8, 6201 SOUTH FREEWAY, FORT WORTH, TX, 76134, US

(21) Appl. No.: 11/761,457

(22) Filed: Jun. 12, 2007

(51) Int. Cl. A61F 9/007

(52) U.S. Cl. 606/107

(57) ABSTRACT

A cartridge for an IOL delivery system that is specifically designed to aid in wound assisted IOL delivery. The distal tip of the nozzle has an extended canopy which serves to open the incision and support the IOL. Peripheral protrusions, flanges, or stops extending laterally from the side of the distal tip provide a positive depth limitation, and prevent the full insertion of the nozzle into the incision. In addition, the protrusion provides support to the incision to reduce the tendency of wound damage through tearing.

This invention relates to intraocular lenses (IOLs) and more particularly to cartridges used to inject IOLs into an eye.

### BACKGROUND OF THE INVENTION

The human eye in its simplest terms functions to provide vision by transmitting and refracting light through a clear outer portion called the cornea, and further focusing the image by way of the lens onto the retina at the back of the eye. The quality of the focused image depends on many factors including the size, shape and length of the eye, and the shape and transparency of the cornea and lens.

When trauma, age or disease cause the lens to become less transparent, vision deteriorates because of the diminished light which can be transmitted to the retina. This deficiency in the lens of the eye is medically known as a cataract. The treatment for this condition is surgical removal of the lens and implantation of an artificial lens or IOL.

While early IOLs were made from hard plastic, such as polymethylmethacrylate (PMMA), soft, foldable IOLs made from silicone, soft acrylics and hydrogels have become increasingly popular because of the ability to fold or roll these soft lenses and insert them through a smaller incision. Several methods of rolling or folding the lenses are used. One popular method is an injector cartridge that folds the lenses and provides a relatively small diameter lumen through which the lens may be pushed into the eye, usually by a soft tip plunger. The most commonly used injector cartridge design is illustrated in U.S. Pat. No. 4,681,102 (Bartell), and includes a split, longitudinally hinged cartridge. Similar designs are illustrated in U.S. Pat. Nos. 5,494,484 and 5,499,987 (Feingold) and U.S. Pat. Nos. 5,616,148 and 5,620,450 (Eagles, et al.). In an attempt to avoid the claims of U.S. Pat. No. 4,681,102, several solid cartridges have been investigated, see for example U.S. Pat. No. 5,275,604 (Rheinish, et al.), U.S. Pat. No. 5,653,715 (Reich, et al.), and U.S. Pat. No. 5,947,976 (Van Noy, et al).

These prior art devices were intended to inject an IOL into the posterior chamber of an aphakic eye through a relatively large (approximately 3.0 mm or larger) incision. Surgical techniques and IOLs have been developed that allow the entire surgical procedure to be performed through much smaller incisions, 2.4 mm and smaller. As a result, surgeons began developing methods of wound assisted IOL insertion, where the IOL is delivered through a small incision without inserting the cartridge tip fully into the wound. In this type of IOL delivery, the wound itself provides a tunnel through which the IOL enters the anterior chamber. Wound assisted IOL delivery, therefore, eliminates the need for the incision to be large enough to accommodate the outer diameter of the cartridge tip, allowing a smaller incision to be used. Prior to the present invention, such wound assisted delivery was accomplished using techniques which are highly reliant on the degree of skill and confidence of the surgeon.

Accordingly, a need continues to exist for an intraocular lens injection cartridge which provides features to specifically aid in wound assisted IOL delivery.

### BRIEF SUMMARY OF THE INVENTION

The present invention improves upon prior art by providing a cartridge for an IOL delivery system that includes an extended canopy at the distal tip of the cartridge to open and support the wound while guiding and controlling the folded lens as it passes through the wound, and a peripheral protrusion, flange, or stop feature that provides an insertion depth limitation and prevention of full insertion of the cartridge tip. In addition, the protrusion provides support to the incision to reduce the tendency of wound damage through tearing.

It is accordingly an objective of the present invention to provide a cartridge for a lens delivery system that has an extended canopy at the distal tip.

It is a further objective of the present invention to provide a cartridge for a lens delivery system that contains a peripheral protrusion, flange, or stop that provides an insertion depth limitation.

It is yet a further objective of the present invention to provide a cartridge for a lens delivery system that contains a peripheral protrusion, flange, or stop that provides support to the incision to reduce the tendency of wound damage through tearing.

Other objectives, features and advantages of the present invention will become apparent with reference to the drawings, and the following description of the drawings and claims.

### BRIEF DESCRIPTION OF THE DRAWINGS

FIG. 1 is an enlarged top perspective view of the lens delivery system cartridge of the present invention.

FIG. 2 is an enlarged front partial perspective view of a first embodiment of the distal tip of the lens delivery system cartridge of the present invention.

FIG. 3 is an enlarged side elevational view of a first embodiment of the distal tip of the lens delivery system cartridge of the present invention.

FIG. 4 is an enlarged front partial perspective view of a second embodiment of the distal tip of the lens delivery system cartridge of the present invention.

FIG. 5 is an enlarged front elevational view of a third embodiment of the distal tip of the lens delivery system cartridge of the present invention.

FIG. 6 is an enlarged side elevational view of the lens delivery system cartridge inserted into an incision in an eye.

### DETAILED DESCRIPTION OF THE PREFERRED EMBODIMENTS

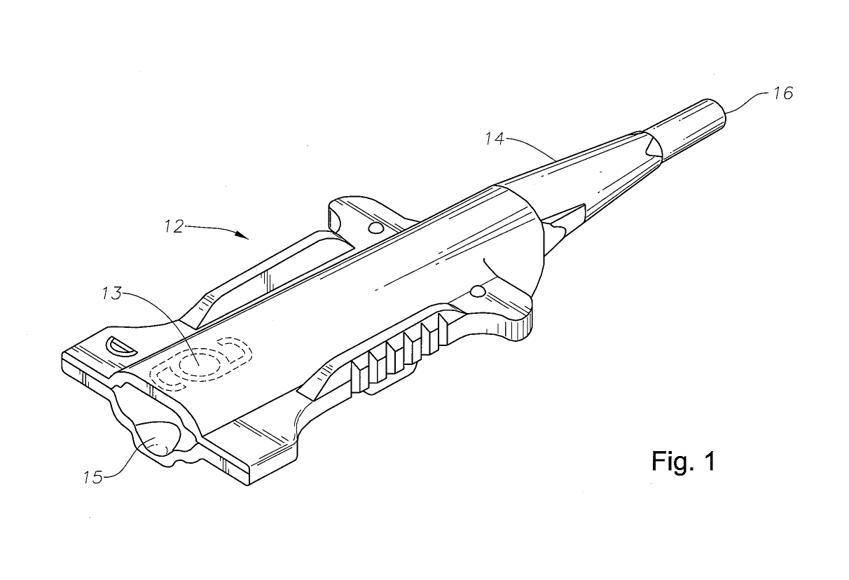

As best seen in FIG. 1, lens cartridge 10 of the present invention generally includes body 12 and nozzle 14. Cartridge 10 can be molded from any suitable thermoplastic, such as polypropylene, and the thermoplastic may contain a lubricity enhancing agent such as those disclosed in U.S. Pat. No. 5,716,364, the entire contents of which are incorporated herein by reference. Nozzle 14 may be integrally formed with body 12. Nozzle 14 includes distal tip 16. Body 12 contains bore or lumen 15. Prior to use, IOL 13 is initially positioned within lumen 15 of body 12.

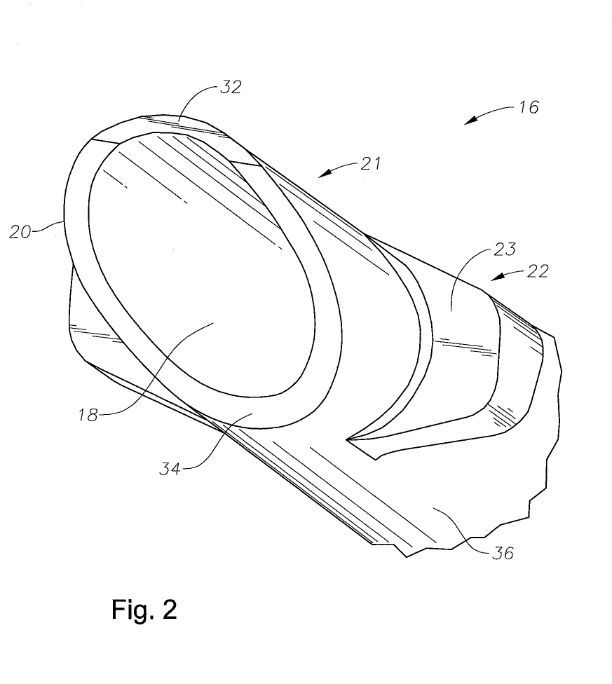

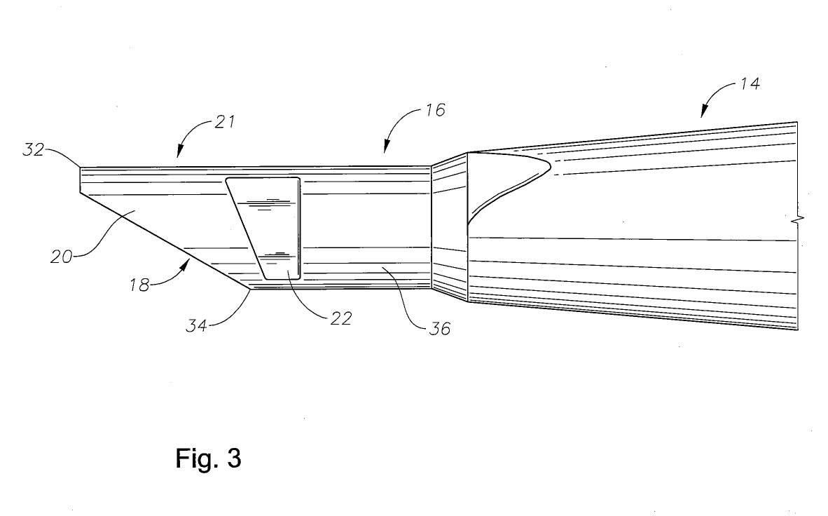

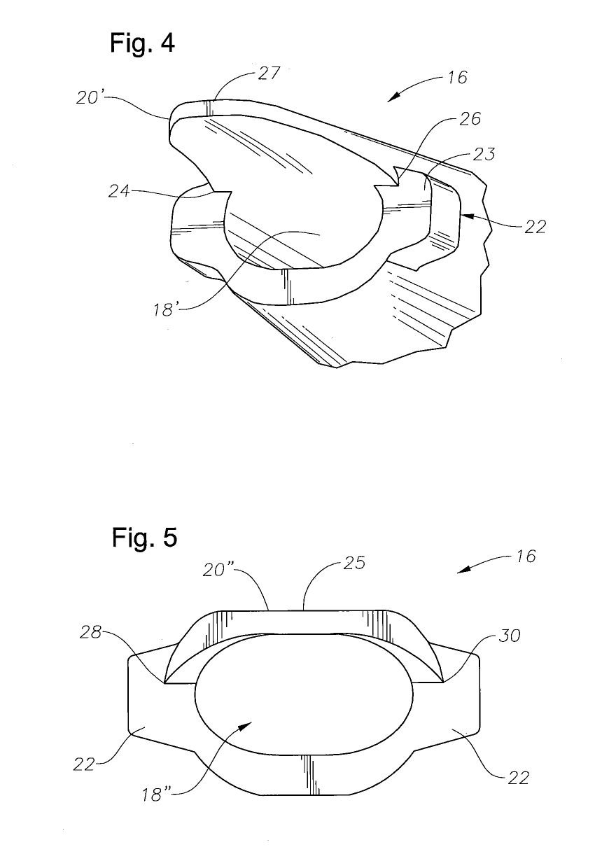

As best seen in FIGS. 2-3, distal tip 16 includes opening 18, canopy 20, and peripheral protrusion 22. Canopy 20 extends from distal tip 16 and functions to open the wound prior to IOL 13 insertion. In addition, upper portion 21 of canopy 20 provides support for the folded IOL 13 during wound assisted delivery. In a preferred embodiment, illustrated in FIGS. 2 and 3, canopy 20 completely encircles opening 18. In this embodiment, canopy 20 gradually tapers from its maximum length of between approximately 1.5 millimeters to 2.4 millimeters (preferably about 1.9 millimeters) at approximately 12 o'clock position 32 to flush with opening 18 at approximately 6 o'clock position 34. In a second embodiment, shown in FIG. 4, canopy 20′ only partially encircles opening 18′ from approximately 10 o'clock position 24 to approximately 2 o'clock position 26. Similarly to canopy 20, canopy 20′ gradually tapers from a maximum length of between approximately 1.5 millimeters to 2.4 millimeters (and preferably about 1.9 millimeters), at position 27, to flush with opening 18′ at approximately 10 o'clock position 24 and approximately 2 o'clock position 26. In a third embodiment, shown in FIG. 5, canopy 20″ substantially encircles opening 18″ from approximately 9 o'clock position 28 to approximately 3 o'clock position 30. In this embodiment, canopy 20″ also gradually tapers from a maximum length of between approximately 1.5 millimeters to 2.4 millimeters (also preferably about 1.9 millimeters), at position 25, to flush with opening 18″ at approximately 9 o'clock position 28 and approximately 3 o'clock position 30.

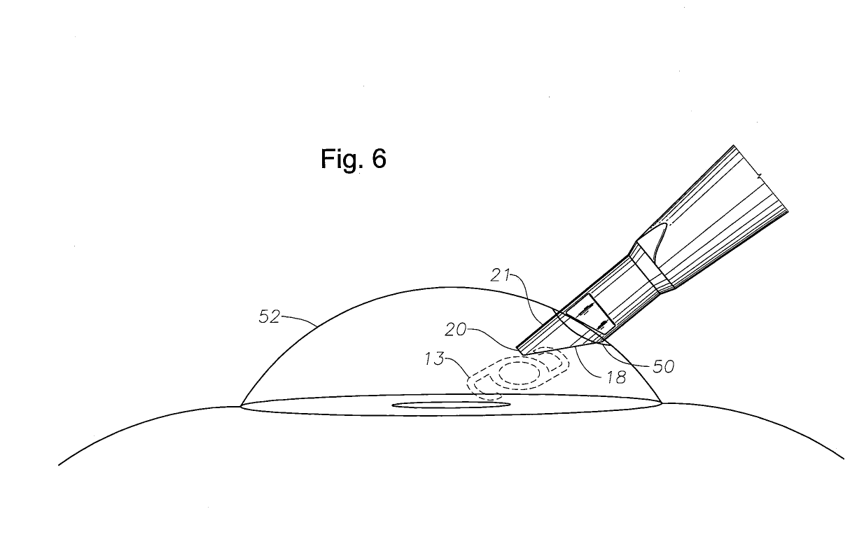

Peripheral protrusion 22 may be any feature appropriate for preventing distal tip 16 from fully entering an incision, such as a flange or stop. Peripheral protrusion 22 extends laterally from outer wall 36 of distal tip 16, and may be a continuous protrusion that completely encircles nozzle 14. However, peripheral protrusion 22 may not be continuous and most preferably comprise a plurality of protrusions 22 that extend, for example, laterally from either side of outer wall 36 of distal tip 16. Peripheral protrusion 22 serves as an insertion depth limitation, and prevents the full insertion of distal tip 16 into the wound entrance. Distal face 23, of peripheral protrusion 22 may be square or sloped at an angle of between approximately 18 to 26 degrees (preferably about 22 degrees). Such a slope will allow a more contoured contact with the surface of the eye and provide for less tissue irritation because cartridge 10 generally is held at an angle to eye 52 during use, as seen in FIG. 6. Both canopy 20 and peripheral protrusion 22 may be integrally formed with distal tip 16 and nozzle 14.

During operation, shown in FIG. 6, the surgeon makes incision 50 in of eye 52. Canopy 20 is inserted into incision 50 to such a depth where peripheral protrusion 22 contacts the surface of eye 52 and prevents further insertion. Upper portion 21 of canopy 20 holds incision 50 open and supports IOL 13 as it is moved through opening 18, through incision 50, and inserted into eye 52.

While certain embodiments of the present invention have been described above, these descriptions are given for purposes of illustration and explanation. Variations, changes, modifications and departures from the systems and methods disclosed above may be adopted without departure from the scope or spirit of the present invention.

We claim:

1. An intraocular lens injector cartridge, comprising:
a) a body having an internal lumen;
b) a tubular nozzle having an outer wall and an opening, the nozzle projecting distally from the body, the opening being fluidly connected to the internal lumen of the body;
c) a canopy projecting distally from the opening; and
d) at least one peripheral protrusion extending laterally from the outer wall of the nozzle proximally from the opening.

2. The cartridge of claim 1 wherein the canopy partially surrounds the opening between approximately a 10 o'clock position to approximately a 2 o'clock position.

3. The cartridge of claim 1 wherein the canopy partially surrounds the opening between approximately a 9 o'clock position to approximately a 3 o'clock position.

4. The cartridge of claim 1 wherein the canopy entirely surrounds the opening.

5. The cartridge of claim 1 wherein the peripheral protrusion comprises a plurality of protrusions.

6. The cartridge of claim 1 wherein the peripheral protrusion comprises a continuous protrusion encircling the nozzle.

7. The cartridge of claim 1 wherein the peripheral protrusion comprises an angled distal face.

8. The cartridge of claim 1 wherein the peripheral protrusion comprises a flange.

9. The cartridge of claim 1 wherein the peripheral protrusion comprises a stop.

10. An intraocular lens injector cartridge, comprising:
a) a body having an internal lumen;
b) a tubular nozzle having an outer wall and an opening, the nozzle projecting distally from the body, the opening being fluidly connected to the internal lumen of the body;
c) a canopy projecting distally from the opening, the canopy partially surrounding the opening from between approximately a 9 o'clock position to approximately a 3 o'clock position; and
d) a plurality of protrusions extending laterally from the outer wall of the nozzle proximally from the opening.

11. The cartridge of claim 10 wherein the protrusions comprise an angled distal face.

12. An intraocular lens injector cartridge, comprising:
a) a body having an internal lumen;
b) a tubular nozzle having an outer wall and an opening, the nozzle projecting distally from the body, the opening being fluidly connected to the internal lumen of the body;
c) a canopy projecting distally from the opening, the canopy partially surrounding the opening from between approximately a 10 o'clock position to approximately a 2 o'clock position; and
d) a plurality of protrusions extending laterally from the outer wall of the nozzle proximally from the opening.

13. The cartridge of claim 12 wherein the protrusions comprise an angled distal face.

14. A method of delivering an intraocular lens to an eye, comprising the steps of:
a) providing an intraocular lens delivery cartridge having a body, a nozzle, and a distal tip, the distal tip having an opening and a canopy protruding distally from the opening, and at least one peripheral protrusion;
b) making an incision into an eye;
c) inserting the canopy of the lens delivery cartridge into the incision;
d) contacting the eye with the peripheral protrusion so as to prevent further insertion of the distal tip into the incision; and
e) inserting the lens through the incision and into the eye.

15. The method of claim 14 further comprising the step of holding the incision open with the canopy.
